(19) United States
(12) Patent Application Publication
(10) Pub. No.: US 2015/0243190 A1
(43) Pub. Date: Aug. 27, 2015

(54) BLOOD PRESSURE MEASUREMENT APPARATUS

(71) Applicant: SEIKO EPSON CORPORATION, Tokyo (JP)

(72) Inventors: Kiyoaki MURAI, Matsumoto-shi (JP); Takuya HIRAIDE, Tatsuno-machi (JP); Michihiro NAGAISHI, Suwa-shi (JP)

(21) Appl. No.: 14/626,276

(22) Filed: Feb. 19, 2015

(30) Foreign Application Priority Data
Feb. 21, 2014 (JP) 2014-031420

(51) Int. Cl. G09B 23/28; A61B 8/00; A61B 8/04

(52) U.S. Cl. CPC G09B 23/28; A61B 8/04; A61B 8/461; A61B 8/4427

(57) ABSTRACT

A blood pressure measurement apparatus includes: a search unit that comes into contact with a living body and receives a signal from the living body; a blood vessel detection section that detects a blood vessel based on the signal; a teaching information generation section that generates teaching information when no blood vessel is detected by the blood vessel detection section at a first site in which the search unit comes into contact with the living body so as to move the search unit in a first direction intersecting the median line of the living body starting from the first site; and a blood pressure calculation section that calculates a blood pressure of the living body based on the signal when the blood vessel is detected by the blood vessel detection section at the first site in which the search unit comes into contact therewith.

### BACKGROUND

1. Technical Field

The present invention relates to a blood pressure measurement apparatus.

2. Related Art

There is a known blood pressure measurement method in which a vascular diameter is measured by using ultrasonic waves and a blood pressure is calculated in accordance with varying vascular diameters. General measurement of a blood vessel adopts a method in which a specialist such as a medical doctor having expert knowledge manipulates an ultrasonic probe of an ultrasonic diagnosis apparatus and a target blood vessel is searched for referring to an ultrasonic image displayed on an image display apparatus. However, in such a method, since a target site such as a blood vessel should be determined referring to the ultrasonic image, and irradiation with ultrasonic waves should be performed in an appropriate direction, it is difficult for a person having no expert knowledge to search for the target site.

In order to solve such a problem, for example, an ultrasonic diagnosis apparatus in which an oscillation mechanism is provided in the ultrasonic probe is proposed (for example, refer to International Publication No. 2011-074271). The ultrasonic probe disclosed in International Publication No. 2011-074271 includes an ultrasonic vibrator array and an oscillation mechanism which oscillates the ultrasonic vibrator array. By oscillating the ultrasonic vibrator array using the oscillation mechanism, measurement can be performed at not only a portion with which the ultrasonic probe is in contact but also a region including the surroundings thereof.

In addition, another ultrasonic diagnosis apparatus in which a manipulator checks an ultrasonic image, a measurement site superimposed on the ultrasonic image, and a schematic diagram of the ultrasonic probe so as to perform positioning of the ultrasonic probe is proposed (for example, refer to International Publication No. 2011-033793). The ultrasonic diagnosis apparatus disclosed in International Publication No. 2011-033793 determines a blood vessel when a blood vessel is present within a measurement range obtained by bringing the ultrasonic probe into contact with a site to be diagnosed. Then, teaching is performed to position the ultrasonic probe with respect to a blood vessel by indicating a positional relationship between a schematic diagram of the determined blood vessel and a schematic diagram of the ultrasonic probe.

Incidentally, if blood pressure measurement using the ultrasonic probe is performed for a long period at a fixed point, an ultrasonic probe which is used by being affixed onto a human body can decrease a load to the manipulator compared to an ultrasonic probe which is used by being grabbed by hand. In order to use the ultrasonic probe being affixed onto a human body, it is desirable that the ultrasonic probe is thinner and smaller-sized than the ultrasonic probe used by being grabbed by hand.

In order to make a blood pressure measurement apparatus able to perform blood pressure measurement at an ordinary household for daily use, it is desirable that the apparatus is easy to handle even for a manipulator having no expert knowledge and can easily search for a blood vessel.

However, in a thinner and smaller-sized ultrasonic probe, it is difficult to provide an oscillation mechanism as the ultrasonic probe adopted in the ultrasonic diagnosis apparatus which is disclosed in International Publication No. 2011/074271, thereby leading to a disadvantage in that the measurement range is narrowed.

In addition, in the ultrasonic diagnosis apparatus and the teaching method of the same disclosed in International Publication No. 2011-033793, when no blood vessel is present within the measurement range of the ultrasonic probe, a manipulator should search for the blood vessel by manipulating the ultrasonic probe while monitoring the ultrasonic image, thereby leading to a disadvantage in that it is difficult for a manipulator having no expert knowledge to search for the blood vessel.

### SUMMARY

An advantage of some aspects of the invention is to solve at least a part of the problems described above, and the invention can be implemented as the following forms or application examples.

### Application Example 1

This application example is directed to a blood pressure measurement apparatus including a search unit that comes into contact with a living body and receives a signal from the living body, a blood vessel detection section that detects a blood vessel based on the signal, a teaching information generation section that generates teaching information when no blood vessel is detected by the blood vessel detection section at a first site in which the search unit comes into contact with the living body so as to move the search unit in a first direction intersecting the median line of the living body starting from the first site, and a blood pressure calculation section that calculates a blood pressure of the living body based on the signal when the blood vessel is detected by the blood vessel detection section at the first site in which the search unit comes into contact therewith.

According to this application example, the blood pressure calculation section calculates the blood pressure when the blood vessel is detected by the blood vessel detection section based on the signal which is received at the first site in which the search unit comes into contact with the living body. When no blood vessel is detected at the first site in which the search unit comes into contact therewith, the teaching information generation section generates teaching information so as to move the search unit from the first site in the direction intersecting the median line.

Major blood vessels (the brachial artery, the carotid artery, the femoral artery, and the like) subjected to blood pressure measurement extend along the median line. Therefore, if the search unit is moved in the first direction intersecting the median line, a movement direction thereof intersects the blood vessels, and thus, a blood vessel can be searched for in less time compared to a case where the ultrasonic probe is moved along the blood vessels. Accordingly, it is possible to provide a blood pressure measurement apparatus in which a blood vessel can be easily detected to measure a blood pressure even though a thinner and smaller-sized ultrasonic probe having a small measurement range is used to be manipulated by a manipulator having no expert knowledge.

### Application Example 2

In the blood pressure measurement apparatus according to the application example described above, it is preferable that when the search unit is moved from the first site based on the teaching information and no blood vessel is detected by the blood vessel detection section at a second site in which the search unit comes into contact with the living body, the teaching information generation section generates teaching information so as to move the search unit in a second direction intersecting the first direction starting from the second site.

According to this application example, when no blood vessel is detected even though the search unit is moved from the first site to the second site based on the teaching information, the teaching information generation section generates teaching information so as to further move the search unit in the second direction intersecting the first direction in which the search unit is moved from the first site to the second site. When no blood vessel is detected even though the ultrasonic probe is moved in the first direction (a direction intersecting the median line), there is a possibility that the first direction is not the direction intersecting a blood vessel. Therefore, in such a case, the ultrasonic probe is further moved in the second direction intersecting the first direction in which the ultrasonic probe is moved from the first site to the second site. Thus, the ultrasonic probe can be moved in the direction intersecting the blood vessel.

### Application Example 3

In the blood pressure measurement apparatus according to the application example described above, it is preferable that an output section is included to be connected to an external device and the teaching information is output to the external device through the output section.

According to this application example, the blood pressure measurement apparatus can output teaching information to an external notification device through the output section, for example. Thus, there is no need to include a notification device in the blood pressure measurement apparatus, thereby making the configuration of the blood pressure measurement apparatus simple. In addition, since selection of the notification device can be made out of various types of devices, convenience for a manipulator is improved.

### Application Example 4

In the blood pressure measurement apparatus according to the application example described above, it is preferable that the blood pressure measurement apparatus includes a notification section that issues a notification of the teaching information.

According to this application example, the blood pressure measurement apparatus includes the notification section that issues a notification of teaching information, and thus, a manipulator of the blood pressure measurement apparatus can move the search unit based on information notified by the notification section.

### BRIEF DESCRIPTION OF THE DRAWINGS

The invention will be described with reference to the accompanying drawings, wherein like numbers reference like elements.

FIG. 1 is a block diagram showing a configuration of a blood pressure measurement apparatus, according to Embodiment 1.

FIGS. 2A and 2B are schematic views showing a configuration of an ultrasonic probe, according to Embodiment 1.

FIGS. 3A and 3B are schematic diagrams showing definitions of directions, according to Embodiment 1.

FIG. 4 is a flowchart illustrating processing of blood pressure measurement performed by the blood pressure measurement apparatus of Embodiment 1, according to Embodiment 1.

FIG. 5 is a schematic diagram showing a teaching form of a contact position generated by a notification device including an image display section, according to Embodiment 1.

FIGS. 6A and 6B are schematic diagrams showing teaching forms of a contact state generated by the notification device including the image display section, according to Embodiment 1.

FIGS. 7A and 7B are schematic diagrams showing a relationship between the contact position and a measurement range of the ultrasonic probe, according to Embodiment 1.

FIGS. 8A and 8B are schematic diagrams showing another relationship between the contact position and the measurement range of the ultrasonic probe, according to Embodiment 1.

FIG. 9 is a schematic diagram showing a movement direction of the ultrasonic probe, according to Embodiment 1.

FIGS. 10A to 10C are schematic diagrams when a portion of a blood vessel is detected within the measurement range of the ultrasonic probe, according to Embodiment 1.

FIGS. 11A to 11C are schematic diagrams when a blood vessel is detected in its entirety within the measurement range of the ultrasonic probe, according to Embodiment 1.

FIGS. 12A to 12C are schematic diagrams when a blood vessel is detected in its entirety within the measurement range of the ultrasonic probe and the measurement range perpendicularly intersects a longitudinal axis direction of a blood vessel, according to Embodiment 1.

FIG. 13 is a schematic view showing a configuration of the ultrasonic probe, according to Modification Example 1.

FIG. 14 is a schematic diagram showing detection of a blood vessel performed by the ultrasonic probe, according to Modification Example 2.

FIGS. 15A to 15C are schematic diagrams illustrating a method of detecting a blood vessel performed by the ultrasonic probe, according to Modification Example 2.

FIGS. 16A to 16C are schematic diagrams illustrating another method of detecting a blood vessel performed by the ultrasonic probe, according to Modification Example 2.

### DESCRIPTION OF EXEMPLARY EMBODIMENTS

Hereinafter, embodiments of the invention will be described with reference to the drawings. The adopted drawings are illustrated in an enlarged, contracted, and exaggerated manner appropriately in order to make portions to be described recognizable. Meanwhile, configurations other than configurations necessary for the description may not be illustrated.

### Embodiment 1

### Configuration of Blood Pressure Measurement Apparatus

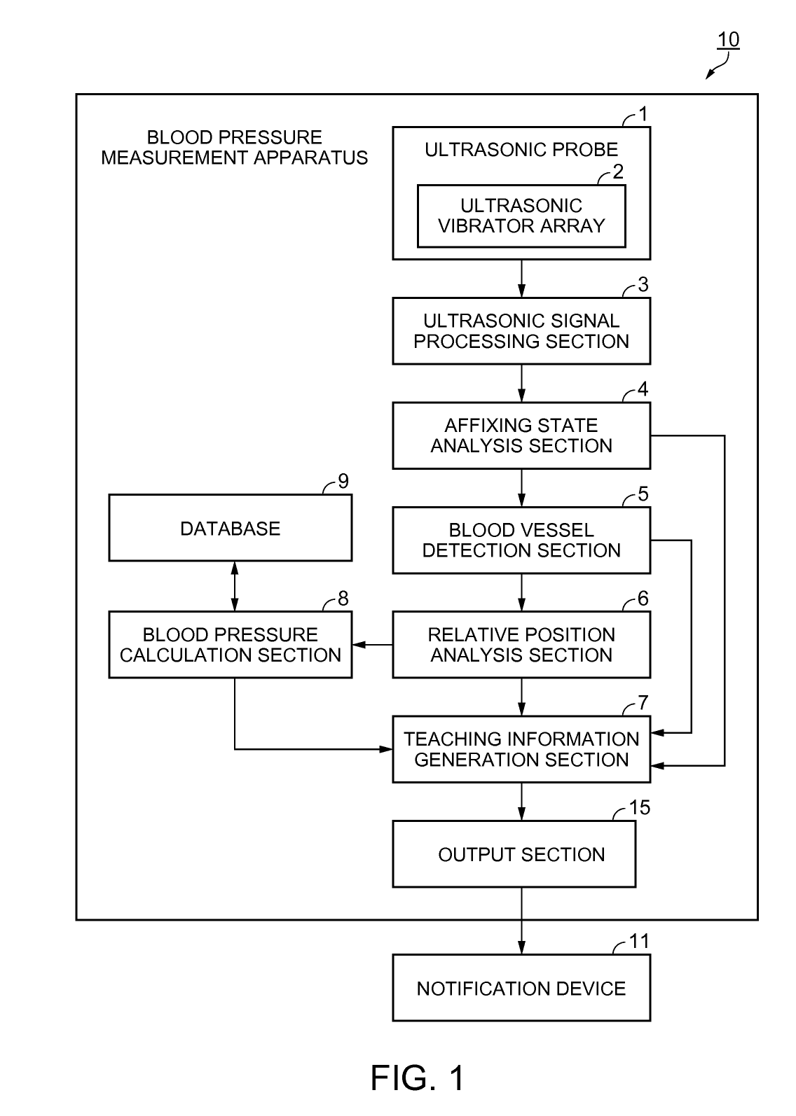

FIG. 1 is a block diagram showing a configuration of a blood pressure measurement apparatus, according to Embodiment 1. Firstly, a schematic configuration of a blood pressure measurement apparatus 10 according to Embodiment 1 will be described.

As shown in FIG. 1, the blood pressure measurement apparatus 10 includes an ultrasonic probe 1 as a search unit, an ultrasonic signal processing section 3, an affixing state analysis section 4, a blood vessel detection section 5, a relative position analysis section 6, a teaching information generation section 7, a blood pressure calculation section 8, and an output section 15. An external notification device 11 to notify a manipulator of teaching information is connected to the blood pressure measurement apparatus 10 through the output section 15.

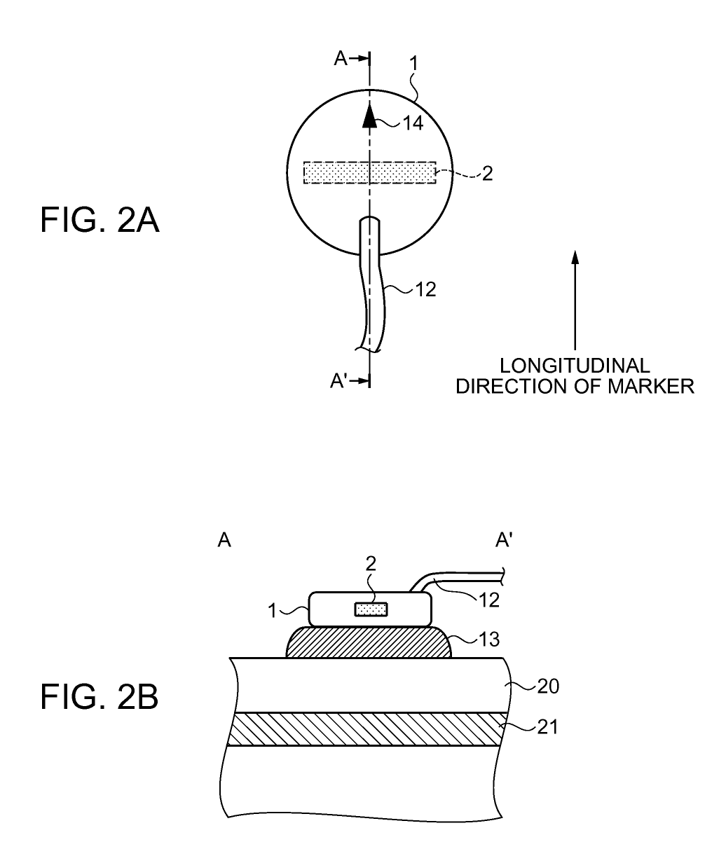

FIGS. 2A and 2B are schematic views showing a configuration of the ultrasonic probe, according to Embodiment 1. In detail, FIG. 2A is a front view of the ultrasonic probe 1 according to Embodiment 1. FIG. 2B is a cross-sectional view taken along line A-A′ in FIG. 2A. FIG. 2B is a schematic diagram showing an affixed form of the ultrasonic probe.

As shown in FIGS. 2A and 2B, the ultrasonic probe 1 has an ultrasonic vibrator array 2 which transceiver ultrasonic waves, and a marker 14. The marker 14 is configured by forming a convex portion or printing in a casing of the ultrasonic probe 1, for example, in order to cause a manipulator to recognize a direction of the ultrasonic probe 1. In the embodiment, the arrowhead-shaped marker 14 is used. Here, a direction indicated by the arrowhead is defined to be a longitudinal axis direction of the marker 14. As the marker 14, something different such as an arrow and multiple dots may be adopted as long as the direction can be easily determined.

The ultrasonic vibrator array 2 is a one-dimensional ultrasonic vibrator array in which a plurality of ultrasonic vibrators are arrayed in a line along one direction, for example. In the embodiment, the direction in which the ultrasonic vibrators are arrayed in a line is referred to as the longitudinal axis direction of the ultrasonic vibrator array 2. The longitudinal axis direction of the ultrasonic vibrator array 2 in the ultrasonic probe 1 is arranged in a direction perpendicular to the longitudinal axis direction of the marker 14.

The ultrasonic probe 1 is connected to the ultrasonic signal processing section 3 (refer to FIG. 1) through a cable 12. The ultrasonic probe 1 has a size which is able to be affixed onto a body surface of a patient 20, and is affixed onto a body surface of the patient 20 by using an adhesion section 13 and the like. The adhesion section 13 is configured with a tape or an adhesive gel, for example. In the blood pressure measurement apparatus 10, when the ultrasonic probe 1 is affixed onto a body surface of the patient 20, an affixing direction of the ultrasonic probe 1 is taught based on the longitudinal axis direction of the marker 14.

The ultrasonic probe 1 can transmit ultrasonic waves from a body surface of the patient 20 as a living body to biological tissue and can receive the ultrasonic waves reflected by the biological tissue, and thus, it is possible to configure a noninvasive blood pressure measurement apparatus 10. The ultrasonic vibrator array 2 can generate various associated waves by having staggered transmission times for ultrasonic waves respectively transmitted by the plurality of ultrasonic vibrators. Then, the ultrasonic vibrator array 2 can vary a transmission angle and a focal length of the associated waves. An ultrasonic wave transmitted from the ultrasonic vibrator array 2 is reflected by a vascular wall inside a living body and is received by the ultrasonic vibrator array 2 as a reflective wave. The reflective wave received by the ultrasonic vibrator array 2 is supplied to the ultrasonic signal processing section 3 through the cable 12 as a signal indicating the reflective wave.

Returning back to FIG. 1, the ultrasonic signal processing section 3 includes a filter, an A/D converter, and the like. The ultrasonic signal processing section 3 calculates a signal indicating the reflective wave which is supplied from the ultrasonic probe 1 through the cable 12 (refer to FIG. 2A). After noise is removed by the filter, a signal transmitted to the ultrasonic signal processing section 3 is supplied to the affixing state analysis section 4 through the A/D converter.

The affixing state analysis section 4 analyzes whether or not the ultrasonic probe 1 is affixed to the patient 20 (refer to FIG. 2B) in a normal contact state. As a result of an analysis, if it is determined that the ultrasonic probe 1 is not in the normal contact state, information indicating an abnormal contact state is supplied from the affixing state analysis section 4 to the teaching information generation section 7 (details will be described later). If it is determined that the ultrasonic probe 1 is in the normal contact state, a signal processed in the ultrasonic signal processing section 3 is supplied to the blood vessel detection section 5.

The blood vessel detection section 5 analyzes whether or not a blood vessel 21 appropriate for blood pressure measurement (refer to FIG. 2B) is detected at a position where the ultrasonic probe 1 is in contact (is affixed). Although details will be described later, as a result of an analysis, if it is determined that the blood vessel 21 appropriate for blood pressure measurement cannot be detected at the position where the ultrasonic probe 1 is in contact, information indicating absence of the blood vessel 21 appropriate for blood pressure measurement is supplied from the blood vessel detection section 5 to the teaching information generation section 7. If it is determined that the blood vessel 21 appropriate for blood pressure measurement is present, a signal processed in the ultrasonic signal processing section 3 is supplied to the relative position analysis section 6.

The relative position analysis section 6 analyzes a relative positional relationship between the blood vessel 21 and the ultrasonic probe 1 used in blood pressure measurement. If the positional relationship between the blood vessel 21 and the ultrasonic probe 1 used in blood pressure measurement is not a relationship appropriate for blood pressure measurement, information indicating the inappropriate positional relationship between the blood vessel 21 and the ultrasonic probe 1 is supplied to the teaching information generation section 7. If the positional relationship between the blood vessel 21 and the ultrasonic probe 1 is in the positional relationship appropriate for blood pressure measurement, a signal processed in the ultrasonic signal processing section 3 is supplied to the blood pressure calculation section 8.

The blood pressure calculation section 8 calculates a blood pressure value based on a signal which is processed in the ultrasonic signal processing section 3. The calculated blood pressure value is supplied to the teaching information generation section 7.

The blood pressure measurement apparatus 10 further includes a database 9. In the database 9, necessary information to calculate a blood pressure value, for example, blood vessel elasticity information of the patient 20 is recorded. Since each patient 20 has different blood vessel elasticity information, blood vessel elasticity information is recorded to be associated with personal information of the patient 20. The database 9 is configured with a hard disk drive or a solid-state drive.

The teaching information generation section 7 generates teaching information and blood pressure information to perform teaching based on the information supplied from each of the affixing state analysis section 4, the blood vessel detection section 5, the relative position analysis section 6, and the blood pressure calculation section 8. The generated information is output outward from the teaching information generation section 7 through the output section 15.

The blood pressure measurement apparatus 10 is connected to the notification device 11 through the output section 15, and the teaching information and the blood pressure information supplied from the teaching information generation section 7 are output to the notification device 11. The connection between the blood pressure measurement apparatus 10 and the notification device 11 through the output section 15 may be either wired connection or wireless connection.

The notification device 11 may be an exclusive terminal or may be a commercially available electronic device such as a smart phone and a tablet PC. Preferably, it is desirable to perform notification by using an image. However, a different method such as audio, an alarm, and blinking by a luminous body may be used as long as a manipulator can understand the notification method. In this manner, if the output section 15 is included in the configuration, the blood pressure measurement apparatus 10 can output teaching information to an external object. Since there is no need to include a notification section, the blood pressure measurement apparatus 10 can have a simple configuration. In addition, since selection of the notification device 11 can be made out of various types of devices, convenience for the manipulator is improved.

In the embodiment, the blood pressure measurement apparatus 10 is configured not to include the notification device 11. However, a notification section may be included in a casing of the blood pressure measurement apparatus 10. In this configuration, even though the notification device 11 is not provided, a manipulator of the blood pressure measurement apparatus 10 can move the ultrasonic probe 1 based on information notified by the notification section.

### Principle of Blood Pressure Calculation

A principle of calculating a blood pressure value by using the blood pressure measurement apparatus 10 (the blood pressure calculation section 8) will be described. The blood pressure value calculated by the blood pressure calculation section 8 is referred to as “a blood pressure value”. Firstly, an ultrasonic wave is transmitted from the ultrasonic vibrator array 2 provided in the ultrasonic probe 1 to the patient 20. Since an ultrasonic wave is reflected by an interface between substances having different acoustic impedances from each other, the ultrasonic wave is reflected by an interface between tissue inside a living body and a vascular wall. The ultrasonic wave is reflected on both sides, a vascular wall (a front wall) on a side close to the ultrasonic probe 1 and a vascular wall (a rear wall) on a side away from the ultrasonic probe 1. The reflective wave reflected by the vascular walls is received by the ultrasonic vibrator array 2, and a difference between the time the reflective wave is received from the front wall and the time the reflective wave is received from the rear wall is measured. The differential time is divided by a velocity of ultrasonic sound in a living body, and thus, a vascular diameter is calculated.

In the embodiment, a blood pressure value is calculated by using a nonlinear function for calculating a blood pressure value from a vascular diameter. In detail, as shown in Expression (1), when a blood pressure value in a blood vessel diastolic phase is Pd, a vascular diameter in the blood vessel diastolic phase is Dd, and a vascular diameter at a certain time is D, a blood pressure value P at a corresponding time is calculated.

$$P = Pd \cdot exp [ β ( \frac{D}{Dd} - I ) ] \qquad ( 1 )$$

A stiffness parameter β in Expression (1) is a coefficient indicating characteristics of elasticity of the blood vessel 21 and is represented by Expression (2). In detail, since a blood pressure value and a vascular diameter have a relationship (P=Dx, here, X is an arbitrary number) of an exponential function, when the blood pressure value P is a natural logarithm (ln(Ps/Pd)) of a ratio of the blood pressure value, and the vascular diameter D is extensibility ((Dd−Ds)/Ds) of an artery wall, the relationship can be shown by a linear line. A slope of the linear becomes β. Ps is a blood pressure value in a blood vessel systolic phase, and Ds is a vascular diameter in the systolic phase. Therefore, the stiffness parameter β represented by Expression (2) needs to be substituted in Expression (1) in order to calculate the blood pressure value by using Expression (1). In addition, the blood pressure value Ps in the blood vessel systolic phase and the blood pressure value Pd in the blood vessel diastolic phase need to be measured in advance before the blood pressure measurement, and the values Ps and Pd are measured by using a cuff-type sphygmomanometer.

$$β = \frac{ln ( \frac{Ps}{Pd} )}{\frac{Ds}{Dd} - I} \qquad ( 2 )$$

### Principle of Blood Vessel Detection

A principle of detecting the blood vessel 21 by using the blood pressure measurement apparatus 10 (the blood vessel detection section 5) will be described. Luminous intensity in an ultrasonic image is utilized to detect the blood vessel 21. To be more specific, a position of the blood vessel is confirmed by observing pulsation of the blood vessel 21 of several pulses in one frame, extracting a portion having luminous intensity from the frame, and determining a scanning line of the extracted portion.

If the blood vessel 21 is detected, discrimination of the blood vessel 21 is performed between an artery and a vein. A peak ratio of velocity in the front wall of the blood vessel is used in discrimination between the artery and the vein. Here, the front wall of the blood vessel denotes the vascular wall on the side close to the ultrasonic probe 1. The discrimination between the artery and the vein is performed by analyzing the peak ratio of velocity in the front wall of the blood vessel, and discrimination is performed based on a peak ratio between a positive component (a component approaching the ultrasonic probe 1) and a negative component (a component being away from the ultrasonic probe 1) in a velocity waveform. Specifically, since the front wall of the artery has a greater velocity of the positive component compared to the front wall of the vein, the front wall of the artery has the greater peak ratio of “the positive component/the negative component”. The front wall of the vein has the peak ratio of “the positive component/the negative component” approximately equal to or smaller than that of the front wall of the artery. The discrimination between the artery and the vein is performed by utilizing the difference between the peak ratios in the velocity waveform.

### Outline of Teaching Method

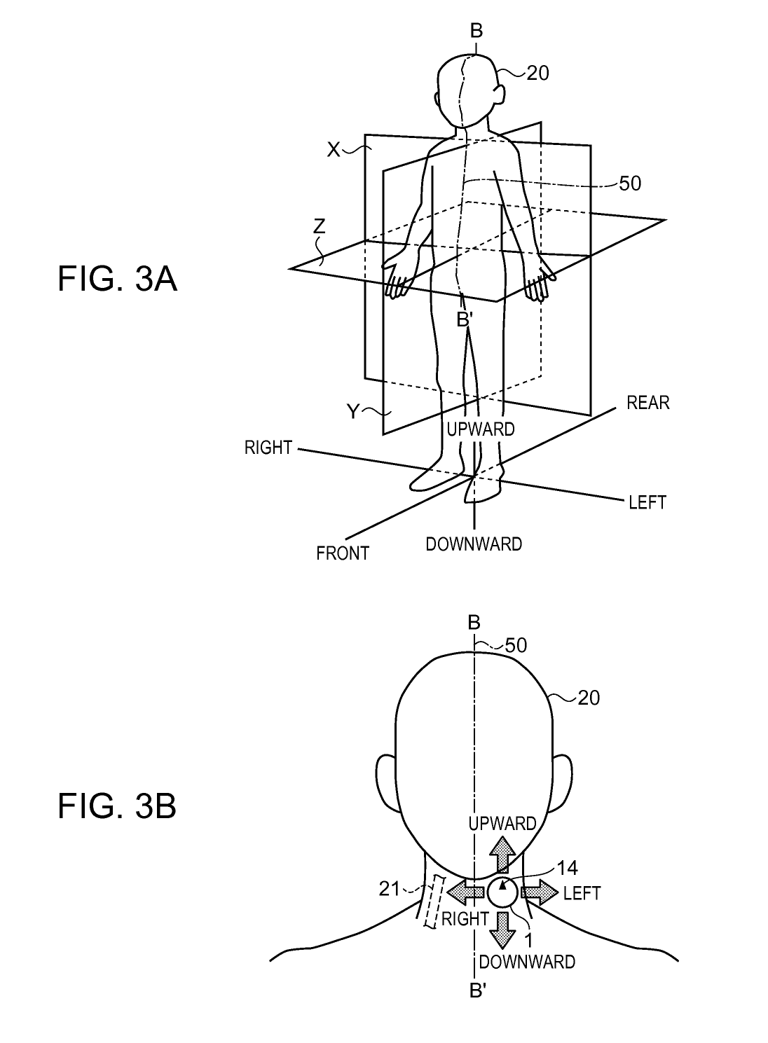

Before describing an operation of the blood pressure measurement apparatus 10, a posture of the patient 20 displayed in the notification device 11 will be defined based on an anatomical definition. FIGS. 3A and 3B are schematic diagrams showing definitions the posture of the patient 20, according to Embodiment 1. FIG. 3A shows an anatomical posture and anatomical planes of the patient 20. The anatomical posture denotes a posture in a state of standing upright, facing forward, stretching both arms downward to the sides of the body, and causing the palm to face the front as illustrated in FIG. 3A. In the embodiment, the anatomical posture is referred to as the basic posture.

As shown in FIG. 3A, the anatomical planes denote three planes such as a forehead plane X, a median plane Y, and a horizontal plane Z. The forehead plane X is a plane dividing the patient 20 into front and rear halves. The front and rear halves are not necessarily limited to be equally divided. The median plane Y is a plane penetrating the patient 20 back and forth to equally divide the patient 20 into right and left halves and is also referred to as a median sagittal plane. The horizontal plane Z is a plane dividing the patient 20 into a head side and a foot side. The horizontal plane Z perpendicularly intersects a longitudinal axis of the patient 20. A line at which the median plane Y and an outer surface of the patient 20 meet (line B-B′ indicated by a dotted line) is defined as a median line 50.

FIG. 3B shows a correlationship between the patient 20 and movement directions of the ultrasonic probe 1. Line B-B′ indicated by a dotted-and-dashed line in FIG. 3B denotes the median line 50 of the patient 20. The arrows in FIG. 3B denote directions which are defined by causing the movement directions of the ultrasonic probe 1 to correspond to the anatomical posture. Here, the term “upward” denotes a direction from the feet to the head while being substantially parallel to an intersection line between the forehead plane X and the median plane Y. The term “downward” denotes a direction from the head to the feet while being substantially parallel to the line intersection between the forehead plane X and the median plane Y. The term “left” denotes a left side direction of the patient 20 while being substantially perpendicular to the median line 50. The term “right” denotes a right side direction of the patient 20 while being substantially perpendicular to the median line 50.

In blood pressure measurement, the blood vessel 21 which is linear compared to capillary blood vessels and extends substantially parallel to the median line 50 is used. As the blood vessel 21, for example, the brachial artery, the carotid artery, and the femoral artery are preferable. However, the blood vessel 21 used in blood pressure measurement is not limited to the above-described blood vessel. A different blood vessel 21 may be used as long as a blood pressure of the blood vessel 21 can be measured. A direction in which the blood vessel 21 extends along the median line 50 is the longitudinal axis of the blood vessel 21. A direction intersecting the longitudinal axis in a substantially perpendicular manner is a short axis of the blood vessel 21. In the embodiment, the carotid artery is used in blood pressure measurement as the blood vessel 21 shown in FIG. 3B.

In the embodiment, the ultrasonic probe 1 is arranged so as to cause the longitudinal axis direction of the marker 14 to be substantially parallel to the median line 50 of the patient 20 (so as to cause the marker 14 to be oriented in an upward direction shown in FIG. 3B), and teaching is performed to move the ultrasonic probe 1 in a direction intersecting the median line 50. As described above, the major blood vessels 21 used in blood pressure measurement are distributed to be less likely to meander, linear, and substantially parallel to the median line 50 compared to other blood vessels. Therefore, the blood vessel 21 can be detected in a short time by performing teaching so as to move the ultrasonic probe 1 in the direction intersecting the median line 50.

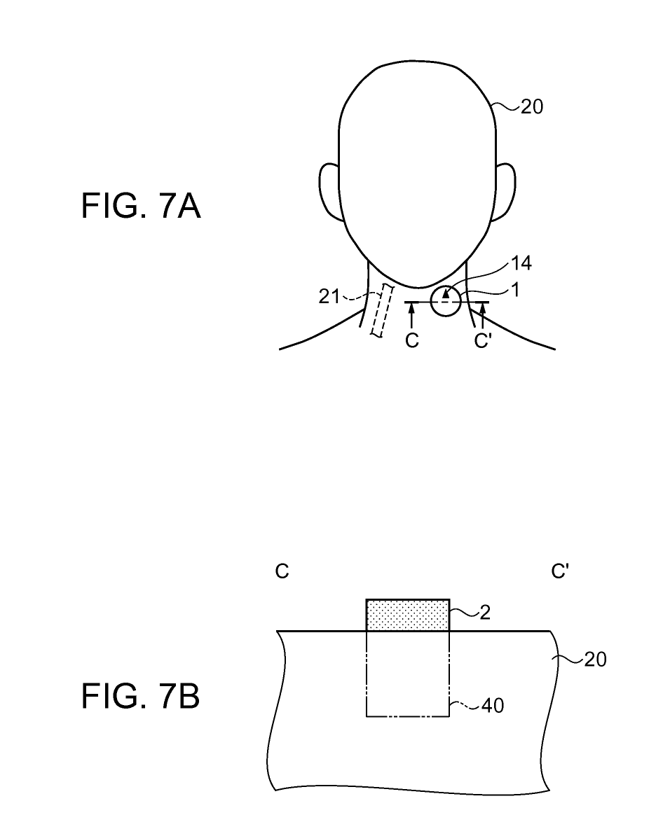

Meanwhile, if teaching is performed so as to move the ultrasonic probe 1 along the longitudinal axis direction of the blood vessel 21, the ultrasonic probe 1 is moved substantially parallel to the longitudinal axis direction of the blood vessel 21. Therefore, when detecting a blood vessel by using the small-sized ultrasonic probe 1 as that in the embodiment, the ultrasonic probe 1 may have to be moved repeatedly for several times in order to detect the blood vessel 21 in a measurement range 40 (refer to FIG. 7B) of the ultrasonic probe 1.

In the embodiment, regardless of the posture of the patient 20 during blood pressure measurement, teaching is performed with the direction corresponding to the above-described anatomical posture. The teaching method of the embodiment is on the assumption that the blood vessel 21 is linear and substantially parallel to the median line 50, and is premised upon the correlationship between an extended site of the blood vessel 21 in the patient 20 and the movement direction of the ultrasonic probe 1.

### Operation of Blood Pressure Measurement Apparatus

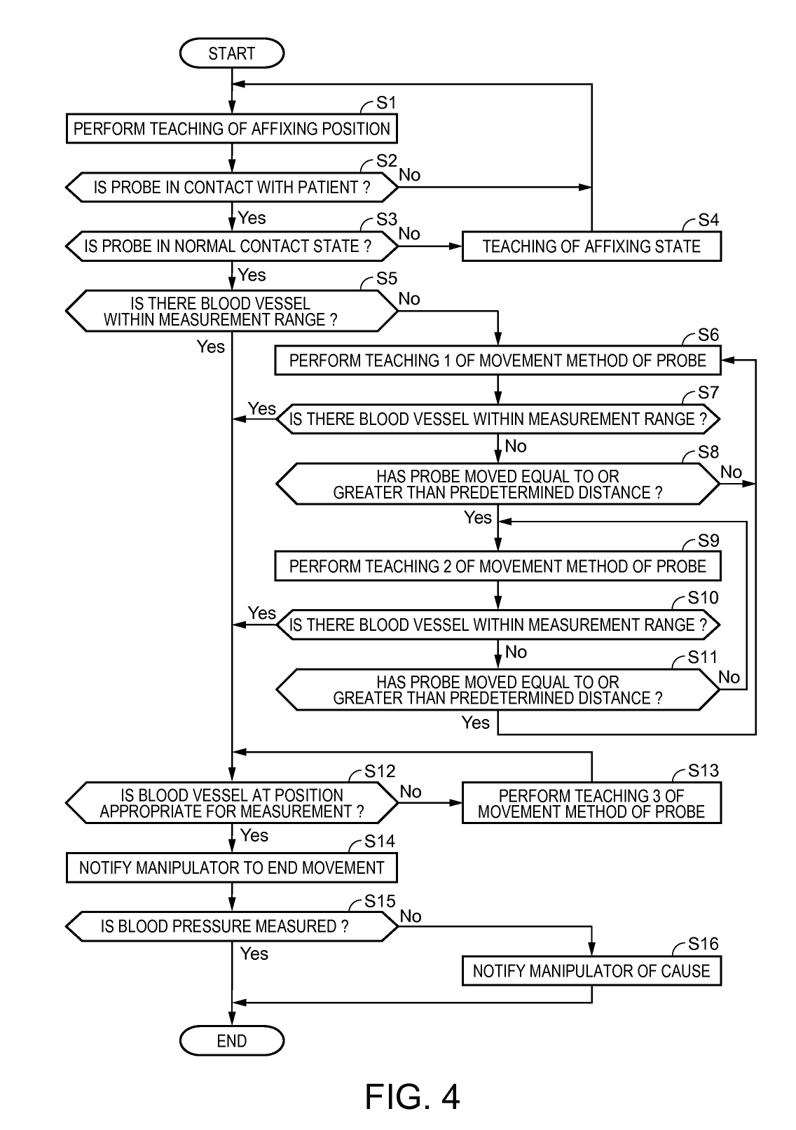

FIG. 4 is a flowchart illustrating processing of blood pressure measurement performed by the blood pressure measurement apparatus, according to Embodiment 1. An operation of the blood pressure measurement apparatus 10 according to the embodiment will be described with reference to FIG. 4. In Step S1 shown in FIG. 4, teaching of an affixing position of the ultrasonic probe 1 is performed.

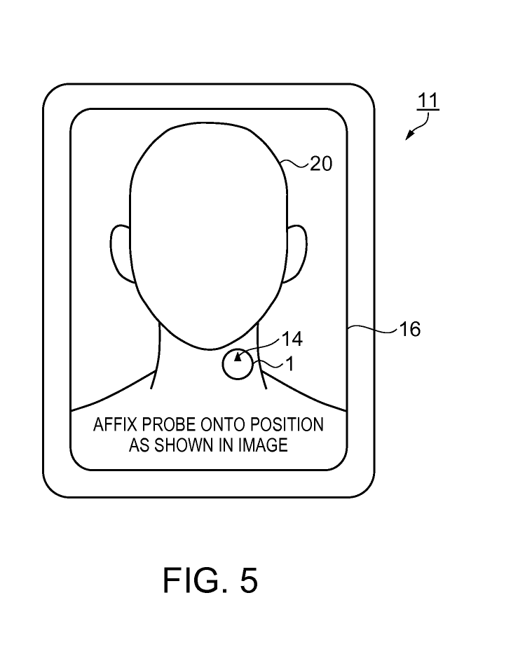

In the embodiment, a method of teaching of the affixing position performed by the notification device 11 which includes an image display section 16 shown in FIG. 5 will be described. FIG. 5 is a schematic diagram showing a teaching form of the affixing position performed by the notification device 11 which includes the image display section 16, according to Embodiment 1. The same reference signs as reference signs applied to real “objects” are applied to reference signs applied to display items in an image displayed on the image display section 16 of the notification device 11 in order to make the description simple. A manipulation button and the like for manipulating the notification device 11 may be arranged in the notification device 11.

As shown in FIG. 5, teaching is performed so as to bring the ultrasonic probe 1 into contact with the left-front side of the neck of the patient 20 as an initial affixing position (a first site) by adopting images and texts displayed on the image display section 16 of the notification device 11. In this manner, a manipulator can visually grasp the affixing position by adopting an image in which the ultrasonic probe 1 is affixed onto the left side of the neck in a front view of the patient 20. In this case, teaching is performed based on the longitudinal axis direction of the marker 14, and thus, the manipulator can easily determine the affixing direction of the ultrasonic probe 1.

Teaching is also performed by adopting texts. Accordingly, it is easier to grasp the affixing position of the ultrasonic probe 1. The embodiment discloses the teaching form in which the ultrasonic probe 1 is affixed onto the left side of the neck. However, an affixing position selection step may be included to select the initial affixing position of the ultrasonic probe 1 out of the right side of the neck, a brachial region, and a femoral region, for example.

Further specified teaching may be performed compared to the above-described teaching of the affixing position. For example, it is possible to adopt an image in which the ultrasonic probe 1 is affixed onto the left side of the neck at a position where a line from the earlobe in a vertically downward direction and a line of the laryngeal prominence in a horizontal direction intersect each other in the front view of the patient 20. A manipulator can grasp the affixing position of the ultrasonic probe 1 more particularly and the following teaching can be easily performed by designating the specific position.

In Step S2 shown in FIG. 4 subsequently to the teaching of the affixing position in Step S1, determination of whether or not the ultrasonic probe 1 is in contact with a body surface of the patient 20 is performed by the affixing state analysis section 4. In the flowchart in FIG. 4, “the ultrasonic probe 1” is simply referred to as “the probe”.

If the ultrasonic probe 1 is in contact with the patient 20, since acoustic impedances of the ultrasonic probe and atmosphere are greatly different from each other, multiple reflections of ultrasonic waves occur in the interface between the ultrasonic probe 1 and atmosphere. A contact state of the ultrasonic probe 1 is determined by analyzing periodic signals generated due to the multiple reflections. Besides, a method of determining the contact state by detecting a body temperature of the patient 20 using a temperature sensor or a method of determining the contact state by detecting BCG using a pressure sensor or a gyro sensor may be adopted.

If it is determined that the ultrasonic probe 1 is not in contact with the patient 20 in Step S2 (Step S2: NO), the procedure returns back to Step S1, thereby successively performing teaching of the affixing position. If it is determined that the ultrasonic probe 1 is in contact with the patient 20 in Step S2 (Step S2: YES), the procedure proceeds to Step S3.

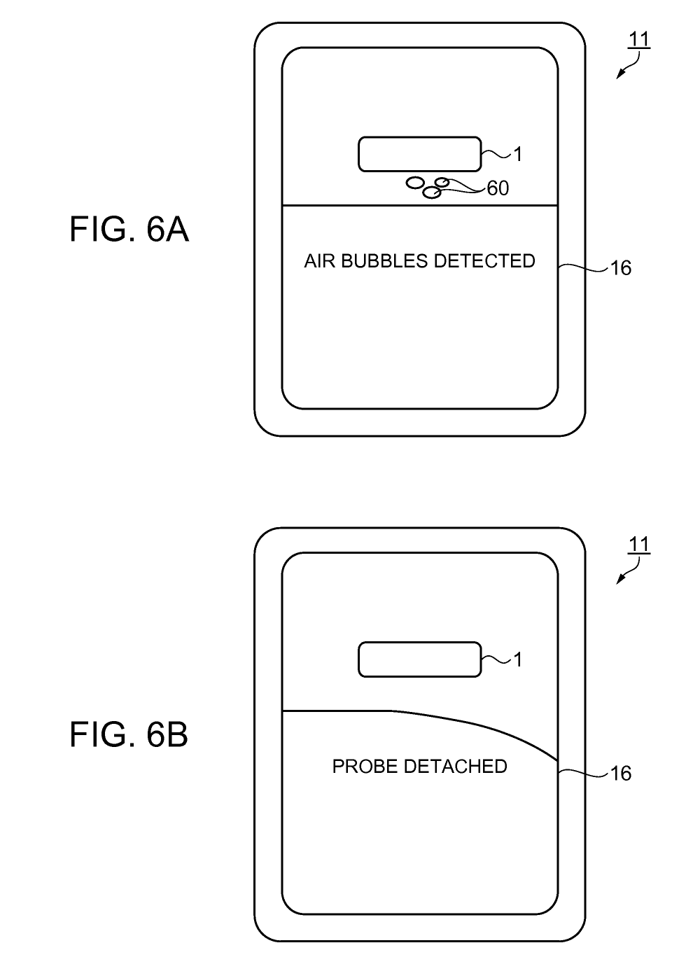

In Step S3, determination of whether or not the ultrasonic probe 1 is in the normal contact state is successively performed in the affixing state analysis section 4. FIGS. 6A and 6B are schematic diagrams showing a teaching form of the contact state generated by the notification device 11 which includes the image display section 16, according to Embodiment 1. FIG. 6A is a schematic diagram showing air bubbles (or foreign materials) 60 intermixed between the ultrasonic probe 1 and the patient 20. FIG. 6B is a schematic diagram showing a portion of the ultrasonic probe 1 detached from the patient 20 (a gap is present between the patient 20 and the ultrasonic probe 1). Both FIGS. 6A and 6B correspond to the cross-sectional views taken along line A-A′ similar to that in FIG. 2B.

The normal contact state of the ultrasonic probe 1 denotes that no air bubble (or no foreign material) 60 (refer to FIG. 6A) or gap (refer to FIG. 6B) is present between the ultrasonic probe 1 and the patient 20. The ultrasonic probe 1 is affixed to the patient 20 by using the adhesion section 13 (refer to FIG. 2B) with a tape or an adhesive gel. In this case, as shown in the schematic diagrams in FIGS. 6A and 6B, if the air bubbles (or the foreign materials) 60 or gap is present between the ultrasonic probe 1 and the patient 20, a great difference of acoustic impedance occurs in the interface with respect to the adhesion section 13 due to the air bubbles (or the foreign materials) 60 or gap, and thus, ultrasonic waves are mostly reflected by the air bubbles (or the foreign materials) 60 or gap. In such a state, it is not possible to obtain signals appropriate for calculating a blood pressure, and thus, determination of whether or not the ultrasonic probe 1 is in the normal contact state is performed by the affixing state analysis section 4.

The reflection of ultrasonic waves caused by the air bubbles (or the foreign materials) 60 or gap is greater than the reflection in the interface between the ultrasonic probe 1 and the adhesion section 13 as well as the reflection in the interface between the adhesion section 13 and a body surface of the patient 20. Since the periodic signals are generated due to multiple reflections in the portion where a great reflection is caused, it is possible to determine whether or not the ultrasonic probe 1 is in the normal contact state by analyzing the periodic signals generated partially in the measurement range 40 of the ultrasonic probe 1. In Step S3, in addition to the above-described method, different methods may be adopted as long as it can be determined whether or not the ultrasonic probe is in the normal contact state through the methods.

If it is determined that the ultrasonic probe 1 is not in normal contact with the patient 20 in Step S3 (Step S3: NO), the procedure proceeds to Step S4, thereby performing teaching of the abnormal contact state.

In Step S4, as shown in FIGS. 6A and 6B, teaching is performed by adopting images and texts through the image display section 16 of the notification device 11 regarding a cause estimated from the received signal. Thereafter, the procedure proceeds to Step S1, thereby repeating teaching of the affixing position. If it is determined that the ultrasonic probe 1 is in normal contact with the patient 20 in Step S3 (Step S3: YES), the procedure proceeds to Step S5.

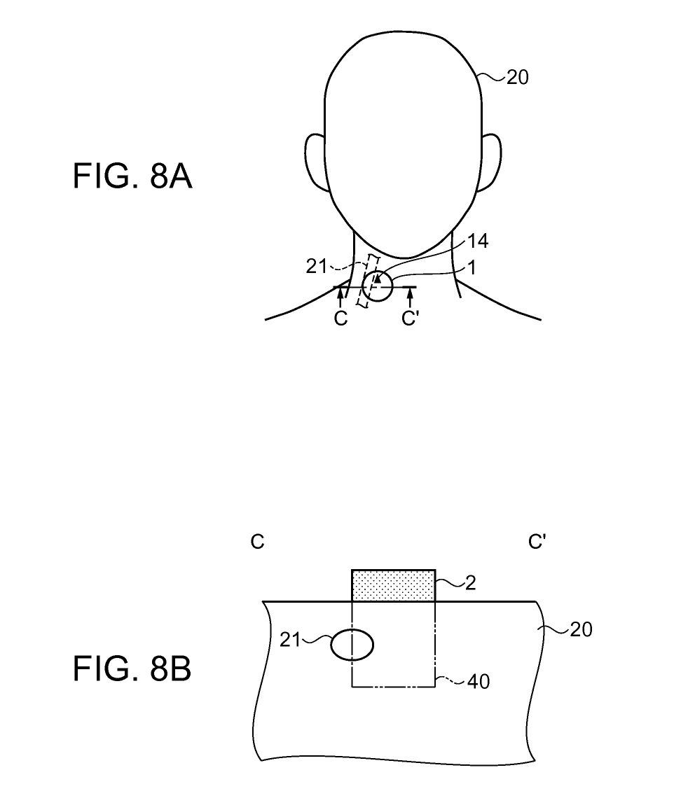

In Step S5, determination of whether or not the blood vessel 21 is detected within the measurement range 40 of the ultrasonic probe 1 is performed by the blood vessel detection section 5. FIGS. 7A and 7B, and FIGS. 8A and 8B are schematic diagrams respectively showing relationships between a position and the measurement range 40 of the ultrasonic probe 1, according to Embodiment 1. FIG. 7A is a plan view showing a positional relationship between the ultrasonic probe 1 and the blood vessel 21, and FIG. 7B is a schematic diagram of a cross section taken along line C-C′ in FIG. 7A. Similarly, FIG. 8A is a plan view showing another positional relationship between the ultrasonic probe 1 and the blood vessel 21, and FIG. 8B is a schematic diagram of a cross section taken along line C-C′ in FIG. 8A.

Lines C-C′ respectively indicated by dotted-and-dashed lines in FIGS. 7A and 8A denotes an array direction of the ultrasonic vibrator array 2. In FIGS. 7B and 8B, the measurement range 40 measured by the ultrasonic vibrator array 2 is shown to be superimposed on a cross section of the patient 20 in the array direction (line C-C′) of the ultrasonic vibrator array 2. FIGS. 7A and 7B are schematic diagrams when the blood vessel 21 is not detected, and FIGS. 8A and 8B are schematic diagrams when the blood vessel 21 is detected.

The blood vessel detection section 5 determines whether or not the blood vessel 21 appropriate for blood pressure measurement is detected within the measurement range 40 of the ultrasonic probe 1. As shown in FIGS. 7A and 7B, if it is determined that the blood vessel 21 appropriate for blood pressure measurement is not detected within the measurement range 40 of the ultrasonic probe 1 (Step S5: NO), the procedure proceeds to Step S6. As shown in FIGS. 8A and 8B, if it is determined that the blood vessel 21 appropriate for blood pressure measurement is detected within the measurement range 40 of the ultrasonic probe 1 (Step S5: YES), the procedure proceeds to Step S12.

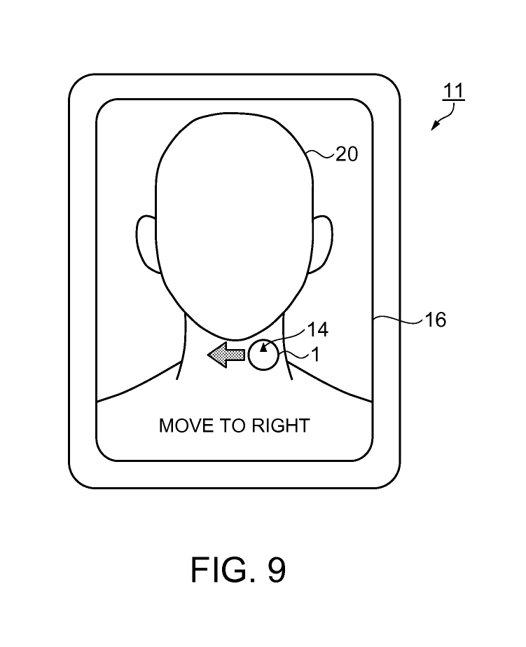

In Step S6, teaching is performed regarding a method of moving the ultrasonic probe 1 to detect the blood vessel 21. FIG. 9 is a schematic diagram showing the movement direction of the ultrasonic probe 1, according to Embodiment 1. In the ultrasonic probe 1, in accordance with teaching in Step S1, the longitudinal axis direction of the marker 14 is substantially parallel to the median line 50 (refer to FIG. 3B) of the patient 20 while being in contact with the left side of the neck. Therefore, teaching is performed so as to move the ultrasonic probe 1 from the initial affixing position (the first site) to the right (a first direction) (Teaching 1). The reason for moving the ultrasonic probe 1 to the right is that if the ultrasonic probe 1 is moved to the left, the ultrasonic probe 1 moves to a rear side where no blood vessel 21 appropriate for blood pressure measurement is present. A method in which teaching of the real movement direction is performed by using arrows, a method in which teaching is performed by adopting texts, or a method in which aforementioned teaching methods are combined is employed in teaching of the movement direction of the ultrasonic probe 1.

If the ultrasonic probe 1 is moved in a right direction in accordance with teaching in Step S6, the procedure proceeds to Step S7, thereby determining whether or not the blood vessel 21 appropriate for blood pressure measurement is detected within the measurement range 40 at a position (a second site) to which the ultrasonic probe 1 is moved, similar to Step S5. In Step S7, as shown in FIGS. 8A and 8B, if it is determined that the blood vessel 21 appropriate for blood pressure measurement is detected within the measurement range 40 of the ultrasonic probe 1 (Step S7: YES), the procedure proceeds to Step S12.

In Step S7, as shown in FIGS. 7A and 7B, if it is determined that the blood vessel 21 appropriate for blood pressure measurement is detected within the measurement range 40 (Step S7: NO), the procedure proceeds to Step S8, thereby estimating a movement distance from the initial affixing position of the ultrasonic probe 1.

As a method of moving the ultrasonic probe 1, the ultrasonic probe 1 may be moved so as to trace a body surface of the patient 20, or the ultrasonic probe 1 may be once separated from the patient 20 and is brought into contact therewith again. In any movement method, a plurality of signals obtained by the blood vessel detection section 5 while moving the ultrasonic probe 1 are combined so as to be recognized as a composite image of the movement direction. The movement distance is estimated from the composite image. If it is determined that the moved distance is equal to or greater than a predetermined value (Step S8: YES), teaching of moving the ultrasonic probe 1 to the right is stopped, and then, the procedure proceeds to Step S9.

The predetermined value of the movement distance of the ultrasonic probe 1 is set based on anatomical distribution of the blood vessel 21, a detection target. For example, the carotid arteries are distributed in the neck one each at the right and left on the front side. Therefore, if the ultrasonic probe 1 is in contact with the position as conducted through teaching in Step S1, the ultrasonic probe 1 traverses the measurement range of the ultrasonic probe 1 as the ultrasonic probe 1 moves half the perimeter of the neck at most. The predetermined value of the movement distance can be specifically decided by providing a step for inputting a height, a weight, a gender, and an affixing position of the patient 20 before performing teaching.

In Step S8, if it is determined that the movement distance of the ultrasonic probe 1 has not reached the predetermined value (Step S8: NO), the procedure returns back to Step S6 again, thereby performing teaching of the movement method of the ultrasonic probe 1.

In Step S9, teaching is performed so as to move the ultrasonic probe 1 in any direction in a vertical direction (a second direction) intersecting a lateral direction (Teaching 2). If the ultrasonic probe 1 is moved in any direction between upward and downward, the procedure proceeds to Step S10, thereby determining whether or not the blood vessel 21 appropriate for blood pressure measurement is detected within the measurement range 40, similar to Step S5. As shown in FIGS. 8A and 8B, if it is determined that the blood vessel 21 appropriate for blood pressure measurement is detected within the measurement range 40 of the ultrasonic probe 1 (Step S10: YES), the procedure proceeds to Step S12.

As shown in FIGS. 7A and 7B, in Step S10, if it is determined that the blood vessel 21 appropriate for blood pressure measurement is detected within the measurement range 40 (Step S10: NO), the procedure proceeds to Step S11, thereby estimating the movement distance from the second site of the ultrasonic probe 1, similar to Step S8.

In Step S11, if it is determined that the movement distance has not reached the predetermined value by comparing the movement distance of the ultrasonic probe 1 to the predetermined value (Step S11: NO), the procedure proceeds to Step S9, thereby continuing teaching so as to move the ultrasonic probe 1.

In Step S11, if it is determined that the ultrasonic probe 1 has moved a distance equal to or greater than the predetermined value (Step S11: YES), the procedure returns back to Step S6 again, thereby performing teaching so as to move the ultrasonic probe 1.

In Step S12, the relative positional relationship between the ultrasonic probe 1 and the blood vessel 21 is determined by the relative position analysis section 6. In blood pressure measurement using ultrasonic waves, measurement accuracy of a blood pressure value depends on measurement accuracy of a diameter of the blood vessel 21. Therefore, it is important that the blood vessel 21 is irradiated with ultrasonic waves perpendicularly to the longitudinal axis direction as much as possible so as to obtain a vascular diameter more accurately. From an analysis result of the relative position analysis section 6, if it is determined that the ultrasonic probe 1 and the blood vessel 21 are not in the positional relationship appropriate for blood pressure measurement (Step S12:NO), the procedure proceeds to Step S13, thereby performing teaching of the method of moving the ultrasonic probe 1 so as to cause the ultrasonic probe 1 to be placed at the position appropriate for blood pressure measurement, with respect to the blood vessel 21 (Teaching 3).

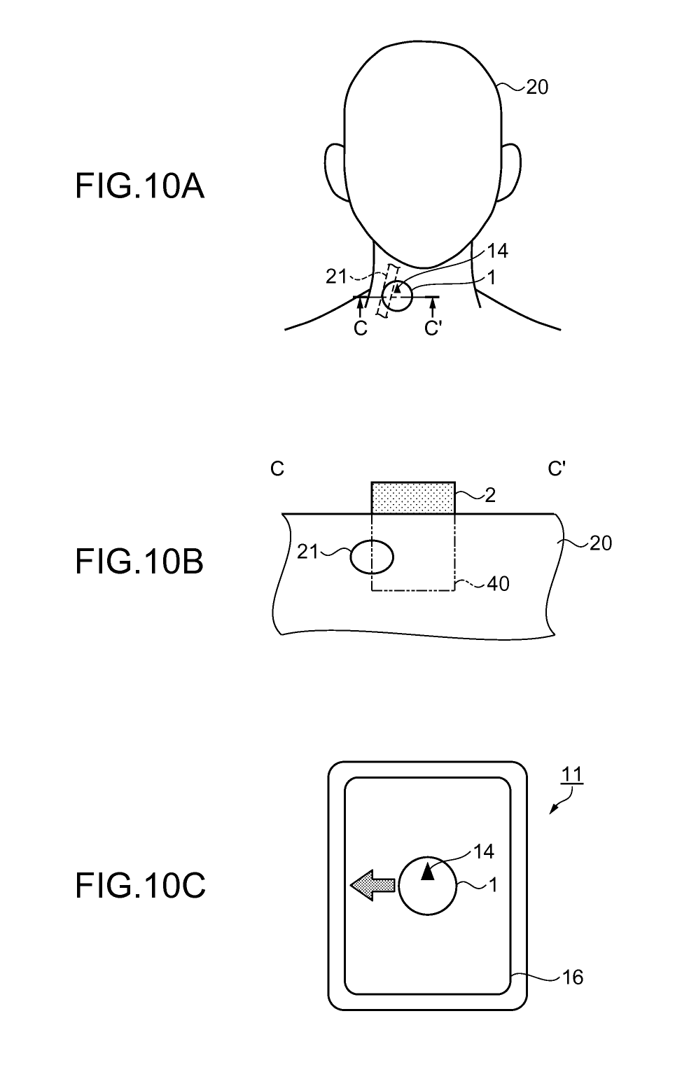

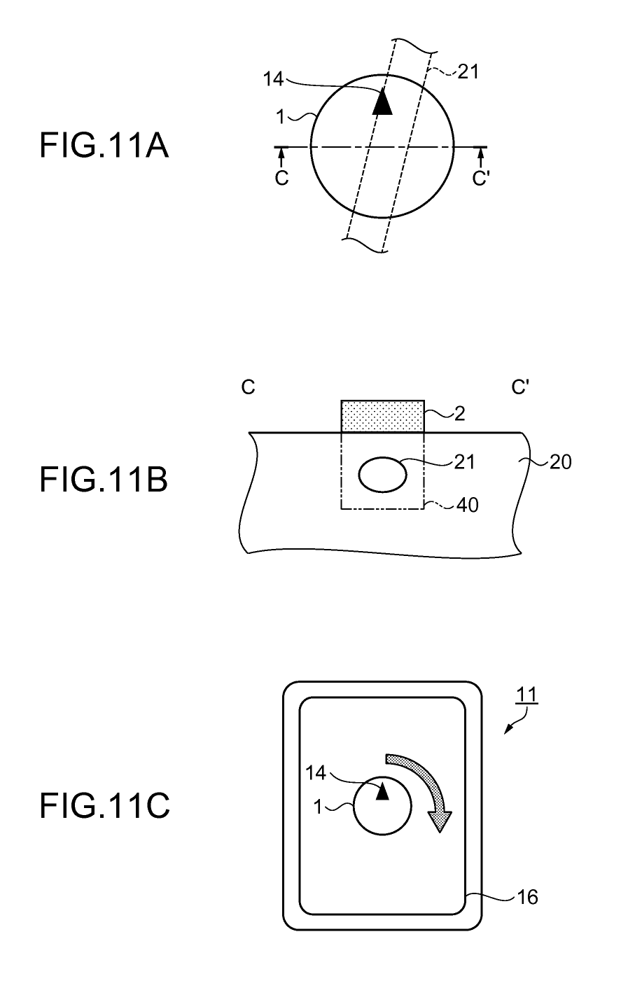

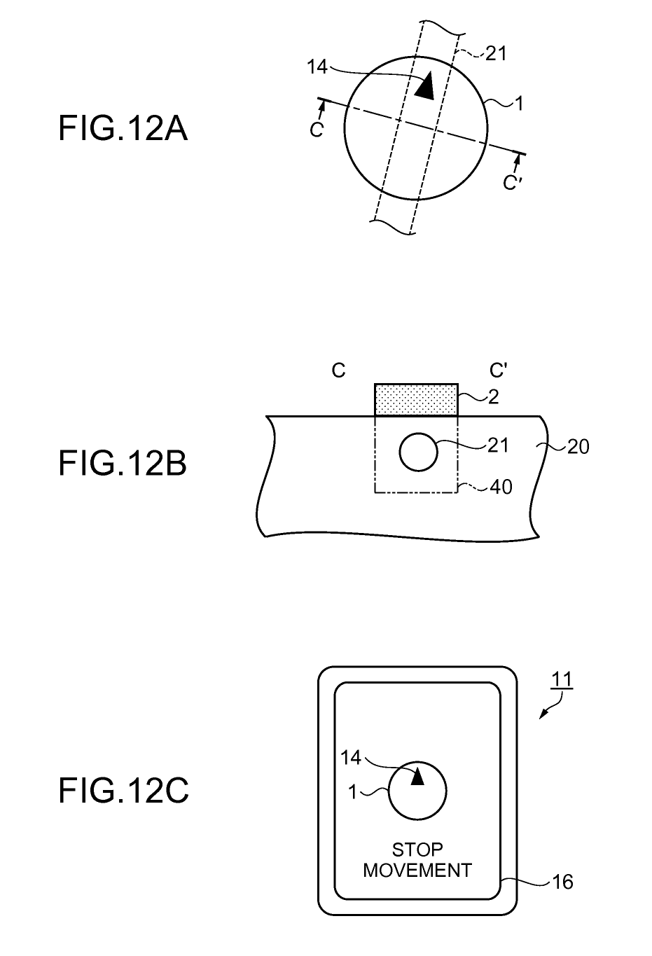

FIGS. 10A to 10C are schematic diagrams when a portion of the blood vessel 21 is detected within the measurement range 40 of the ultrasonic probe 1, according to Embodiment 1. FIGS. 11A to 11C are schematic diagrams when the blood vessel 21 is detected in its entirety within the measurement range 40 of the ultrasonic probe 1, according to Embodiment 1. FIGS. 12A to 12C are schematic diagrams when the blood vessel 21 is detected in its entirety within the measurement range 40 of the ultrasonic probe 1 and the measurement range 40 perpendicularly intersects the longitudinal axis direction of a blood vessel 21, according to Embodiment 1. Specifically, FIGS. 10A, 11A, and 12A are schematic diagrams respectively showing positional relationships between the blood vessel 21 and the ultrasonic probe 1 in each case. FIGS. 10B, 11B, and 12B are schematic diagrams respectively showing assumed cross sections of the measurement range 40 in each case. FIGS. 10C, 11C, and 12C are schematic diagrams showing teaching images of the notification device 11 in each case. FIGS. 11A and 12A show the enlarged ultrasonic probe 1.

When a portion of the blood vessel 21 is detected as shown in FIGS. 10A and 10B, a manipulation in FIG. 10C is performed so that the cross section of the blood vessel 21 is detected in its entirety as shown in FIGS. 11A and 11B. Subsequently, when the cross section of the blood vessel 21 is rendered in its entirety as shown in FIGS. 11A and 11B, a manipulation in FIG. 11C is performed so that the cross section of the blood vessel 21 is detected in its entirety and the measurement range 40 perpendicularly intersects the longitudinal axis direction of a blood vessel 21 as shown in FIGS. 12A and 12B. A manipulation shown in FIG. 12C is performed when the cross section of the blood vessel 21 is detected in its entirety and the measurement range 40 perpendicularly intersects the longitudinal axis direction of a blood vessel 21 as shown in FIGS. 12A and 12B.

FIGS. 10A and 10B show that the blood vessel 21 is detected in Step S5, Step S7, or Step S10. In such a state, it is difficult to accurately measure the vascular diameter. Therefore, it is necessary to move the ultrasonic probe 1 in Step S13 and to cause the center of the ultrasonic probe 1 to overlap with the blood vessel 21 as shown in FIGS. 11A and 11B.

In a case of FIGS. 10A and 10B, since the center of the ultrasonic probe 1 can overlap with the blood vessel 21 by moving the ultrasonic probe 1 to the right, teaching is performed on the image display section 16 of the notification device 11 as shown in FIG. 10C so as to move the ultrasonic probe 1 to the right based on teaching information from the teaching information generation section 7. Teaching shown in FIG. 10C is performed until the center of the ultrasonic probe 1 overlaps with the blood vessel 21. In the meantime, Steps S13 and S12 are repeatedly performed.

In Step S12, if it is determined that the center of the ultrasonic probe 1 overlaps with the blood vessel 21, the procedure proceeds to Step S13 again. In this case, teaching is performed to rotate the ultrasonic probe 1 so as to be shifted from a state where the center of the ultrasonic probe 1 overlaps with the blood vessel 21 as shown in FIGS. 11A and 11B to a state where the direction of the marker 14 substantially coincides with the longitudinal axis direction of a blood vessel 21 as shown in FIGS. 12A and 12B.

In this case, teaching is performed in the notification device 11 so as to rotate the ultrasonic probe 1 clockwise as shown in FIG. 11C based on teaching information from the teaching information generation section 7. Teaching shown in FIG. 11C is performed until the longitudinal axis direction of the marker 14 substantially coincides with the longitudinal axis direction of a blood vessel 21. In the meantime, Steps S13 and S12 are repeatedly performed.

In Step S12, as in FIGS. 12A and 12B, if it is determined that the longitudinal axis direction of the marker substantially coincides with the longitudinal axis direction of a blood vessel 21, the procedure proceeds to Step S14, thereby performing teaching in the notification device 11 so as to end the movement of the ultrasonic probe 1 as shown in FIG. 12C based on teaching information from the teaching information generation section 7.

Thus far, descriptions have been given regarding positioning in the order of detecting a portion of the blood vessel 21 (FIGS. 10A and 10B), overlapping the center of the ultrasonic probe 1 with the blood vessel 21 (FIGS. 11A and 11B), and causing the longitudinal axis direction of the marker 14 to substantially coincide with the longitudinal axis direction of a blood vessel 21 (FIGS. 12A and 12B). However, the teaching method of the blood pressure measurement apparatus 10 according to the embodiment is not limited to such forms. For example, in a case where the center of the ultrasonic probe 1 overlaps with the blood vessel 21 (FIGS. 11A and 11B) or in a case where the longitudinal axis direction of the marker 14 substantially coincides with the longitudinal axis direction of a blood vessel 21 (FIGS. 12A and 12B) when the blood vessel 21 is detected in Step S5, appropriate teaching is performed for each state.

Subsequently, in Step S15, calculation of a blood pressure starts by the blood pressure calculation section 8. If calculation of a blood pressure starts and it is determined that calculation of a blood pressure value is in process (Step S15: YES), teaching processing ends. If it is determined that calculation of a blood pressure value is not in process (Step S15: NO), the procedure proceeds to Step S16, and a cause hindering calculation of a blood pressure value is analyzed to be notified of the cause, thereby ending the teaching processing. If the cause hindering calculation of a blood pressure value cannot be analyzed, notification of a failure is generated, thereby ending teaching processing.

In the embodiment, teaching is performed so as to move the ultrasonic probe 1 in the lateral direction (a direction intersecting the median line) in Step S6, and to move the ultrasonic probe 1 in the vertical direction in Step S9 when it is determined that no blood vessel 21 is present. However, the movement direction in Step S6 may be a direction different from the above-described direction as long as the direction intersects the median line 50. The movement direction in Step S9 may be a direction different from the above-described direction as long as the direction intersects the movement direction in Step S6.

In the embodiment, teaching is performed by adopting images and texts. However, teaching may be performed by a different method such as audio in place of images and texts as long as the method allows a manipulator to recognize the method of moving the ultrasonic probe 1.

As described above, according to the blood pressure measurement apparatus 10 of the embodiment, it is possible to obtain the following effects.

By adopting the configuration of the blood pressure measurement apparatus 10 according to the embodiment, even though the ultrasonic probe 1 searching for the blood vessel 21 is in contact with a position where no blood vessel 21 is present, the teaching information generation section 7 generates teaching information so as to cause the ultrasonic probe 1 to move from a site of the patient 20 with whom the ultrasonic probe 1 comes into contact in the direction intersecting the median line 50. Accordingly, it is possible to provide the blood pressure measurement apparatus 10 which can search for the blood vessel 21 in a short time. Moreover, by adopting the configuration of the embodiment, even though the ultrasonic probe 1 is moved in the direction intersecting the median line 50 and it is determined that no blood vessel 21 is present, the blood vessel 21 is continuously searched for, and thus, the blood vessel 21 can be reliably detected.

Since the longitudinal axis direction of the marker 14 and the longitudinal axis direction of the ultrasonic vibrator array 2 are configured to perpendicularly intersect each other, a short axis direction of the blood vessel 21 is detected by laterally moving the ultrasonic probe 1. Accordingly, it is possible to be shifted to measurement of the vascular diameter by only performing a fine adjustment for a position of the ultrasonic probe 1, and thus, blood pressure measurement can be promptly performed. In addition, since a manipulator can perform positioning of the ultrasonic probe 1 with respect to the blood vessel 21 without referring to an ultrasonic image, positioning thereof can be easily performed by using the thinner and smaller-sized ultrasonic probe 1 even though the manipulation is performed by a manipulator having no expert knowledge. Moreover, according to the blood pressure measurement apparatus 10 of the embodiment, the patient 20 oneself can be a manipulator moving the ultrasonic probe 1.

The invention is not limited to the above-described embodiment, and various changes and modifications can be added to the above-described embodiment. The following are modification examples.

### Modification Example 1

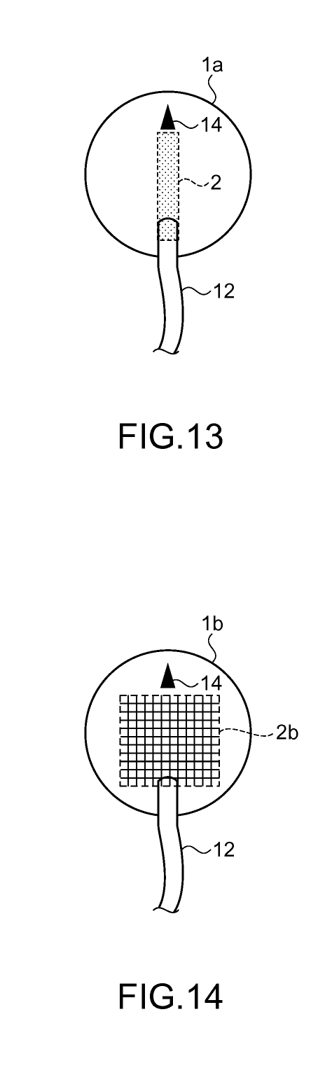

In Embodiment 1, as shown in FIGS. 2A and 2B, the longitudinal axis direction of the ultrasonic vibrator array 2 is configured to be perpendicular to the longitudinal axis direction of the marker 14, in the description. However, the invention is not limited to such a form. FIG. 13 is a schematic view showing a configuration of the ultrasonic probe, according to Modification Example 1. Hereinafter, an ultrasonic probe 1a according to Modification Example 1 will be described. The same reference numerals and signs are applied to sections and portions having the same configuration as Embodiment 1, and the overlapping descriptions will not be repeated.

FIG. 13 shows the ultrasonic probe 1a in which the longitudinal axis direction of the ultrasonic vibrator array 2 (one-dimensional ultrasonic vibrator array) is configured to be parallel to the longitudinal axis direction of the marker 14. As shown in FIG. 13, if the longitudinal axis direction of the marker 14 and the longitudinal axis direction of the ultrasonic vibrator array 2 are configured to be parallel to each other, the longitudinal axis direction of a blood vessel 21 is detected by moving the ultrasonic probe 1a in the lateral direction. Since multiple vascular walls can be selected when detecting the blood vessel 21 in the longitudinal axis direction, it is easier than detecting a blood vessel in the short axis direction. Thus, a blood vessel can be smoothly detected.

According to the configuration of the ultrasonic probe 1a of Modification Example 1, since the ultrasonic vibrator array 2 is arranged to be parallel to the longitudinal axis direction of the marker 14, the longitudinal axis direction of the blood vessel 21 can be detected by arranging the longitudinal axis direction of the marker 14 to be substantially parallel to the median line 50. Thus, it is possible to obtain the effect similar to that of the blood pressure measurement apparatus 10 of Embodiment 1.

Through Embodiment 1 and Modification Example 1, the cases where the longitudinal axis direction of the ultrasonic vibrator array 2 and the longitudinal axis direction of the marker 14 are arranged to be perpendicular and parallel to each other have been described. However, an angle formed by the longitudinal axis direction of the marker 14 and the longitudinal axis direction of the ultrasonic vibrator array 2 may be an angle other than being perpendicular or parallel. By having such a configuration, it is also possible to obtain the effect similar to that in blood vessel detection in which the ultrasonic probe 1 and the ultrasonic probe 1a moves in the lateral direction.

### Modification Example 2

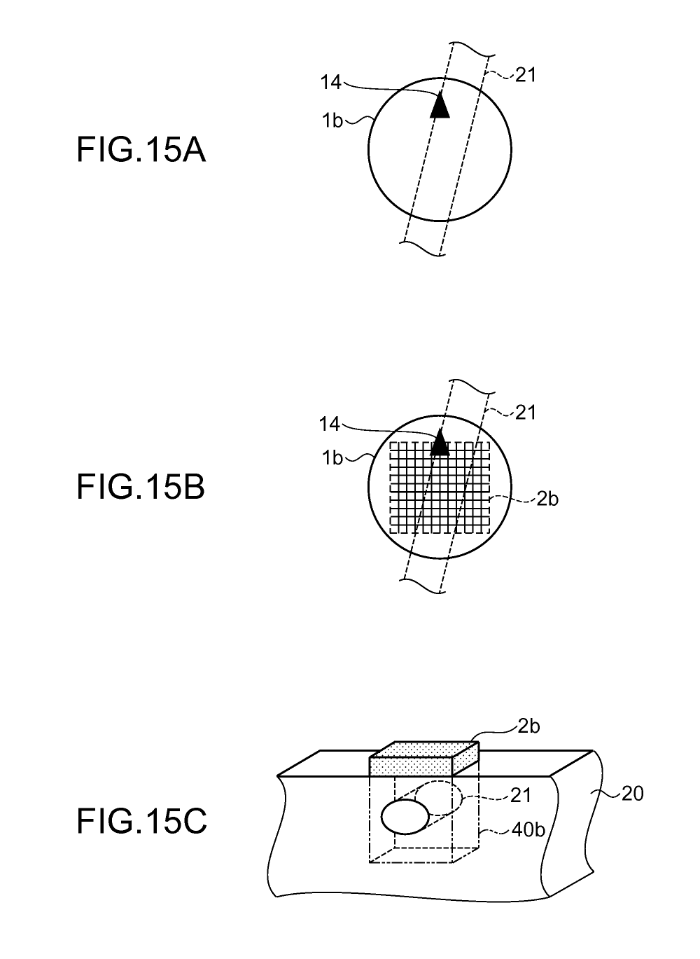

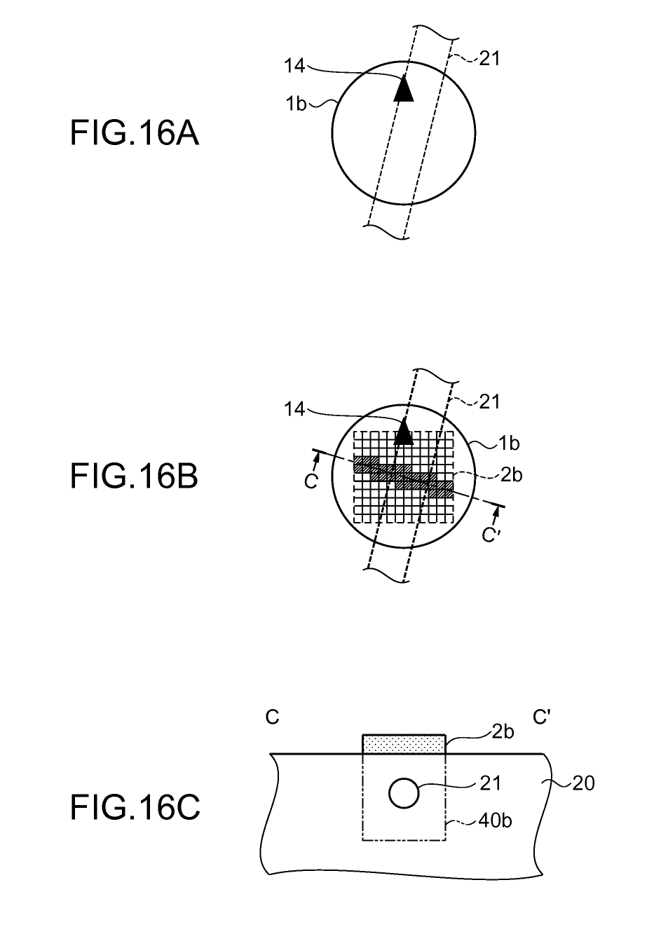

In Embodiment 1 and Modification Example 1, the ultrasonic probes 1 and 1a having the ultrasonic vibrator array in which ultrasonic vibrators are arrayed in a one-dimensional manner are respectively adopted. However, the invention is not limited to such a form. FIG. 14 is a schematic view showing a configuration of the ultrasonic probe, according to Modification Example 2. FIGS. 15A to 15C and FIGS. 16A to 16C are schematic diagrams showing a method of detecting a blood vessel performed by the ultrasonic probe, according to Modification Example 2.

An ultrasonic vibrator array 2b shown in FIG. 14 has the ultrasonic vibrators which are configured to be arrayed in a two-dimensional manner. In Modification Example 2, an ultrasonic probe 1b having the ultrasonic vibrator array 2b in which the ultrasonic vibrators are arrayed in the two-dimensional manner will be described. In order to make the description simple, the description will be given from a state where the center of the ultrasonic probe 1b already overlaps with the blood vessel 21.

FIGS. 15A and 16A show positional relationships when the center of the ultrasonic probe 1b overlaps with the blood vessel 21. FIGS. 15B and 16B are schematic diagrams of the ultrasonic vibrator configuring the ultrasonic vibrator array 2b. FIGS. 15C and 16C are schematic diagrams showing a measurement range 40b of the ultrasonic probe 1 and a blood vessel being detected.

In a case of the ultrasonic vibrator array 2b in which the ultrasonic vibrators are arrayed in a two-dimensional manner, when detecting the blood vessel 21 in Steps S5 and S10 shown in FIG. 4, all the ultrasonic vibrators in the ultrasonic vibrator array 2b are used as shown in FIG. 15B. Therefore, the ultrasonic probe 1b has the three-dimensional measurement range 40b as shown in FIG. 15C.

If the blood vessel 21 is detected in Step S5 or S10, the procedure proceeds to Step S12. In Step S12 of Modification Example 2, unlike Step S12 of Embodiment 1, when positioning is performed so as to cause the center of the ultrasonic probe 1b to overlap with the blood vessel 21 as shown in FIG. 15A, positioning ends without performing positioning of the ultrasonic probe 1b in a rotation direction (refer to FIG. 16A).

If positioning ends in Step S12, the ultrasonic vibrators necessary to perform blood pressure measurement are selected by the relative position analysis section 6 out of the ultrasonic vibrator array 2b. Then, the ultrasonic vibrators for obtaining a cross section of the blood vessel 21 in the short axis direction is selected as shown in FIG. 16B. In FIG. 16B, the selected ultrasonic vibrators out of the ultrasonic vibrator array 2b are indicated by being applied with oblique lines.

In this manner, in Modification Example 2, in the ultrasonic vibrator array 2b in which the ultrasonic vibrators are arrayed in the two-dimensional manner, since the ultrasonic vibrators used by the relative position analysis section 6 is selected in accordance with a positional relationship between the blood vessel 21 and the ultrasonic probe 1b, positioning of the ultrasonic probe 1b in the rotation direction is no longer necessary. Therefore, a manipulator of the blood pressure measurement apparatus 10 can further easily perform positioning of the ultrasonic probe 1 at a position appropriate for measuring the blood vessel 21.

As described above, according to the configuration of the ultrasonic probe 1b of Modification Example 2, since positioning of the ultrasonic probe 1b in the rotation direction is no longer necessary by providing the configuration including the ultrasonic vibrator array 2b having a two-dimensional arrangement, a manipulator can further easily perform positioning of the ultrasonic probe 1b, in addition to the effects in the blood pressure measurement apparatus 10 of Embodiment 1.

The entire disclosure of Japanese Patent Application No. 2014-031420, filed Feb. 21, 2014 is expressly incorporated by reference herein.

What is claimed is:

1. A blood pressure measurement apparatus, comprising:
a search unit that comes into contact with a living body and receives a signal from the living body;
a blood vessel detection section that detects a blood vessel based on the signal;
a teaching information generation section that generates teaching information when no blood vessel is detected by the blood vessel detection section at a first site in which the search unit comes into contact with the living body so as to move the search unit in a first direction intersecting the median line of the living body starting from the first site; and
a blood pressure calculation section that calculates a blood pressure of the living body based on the signal when the blood vessel is detected by the blood vessel detection section at the first site in which the search unit comes into contact therewith.

2. The blood pressure measurement apparatus according to claim 1,
wherein when the search unit is moved from the first site based on the teaching information and no blood vessel is detected by the blood vessel detection section at a second site in which the search unit comes into contact with the living body, the teaching information generation section generates teaching information so as to move the search unit in a second direction intersecting the first direction starting from the second site.

3. The blood pressure measurement apparatus according to claim 1, further comprising:
an output section that is to be connected to an external device,
wherein the teaching information is output to the external device through the output section.

4. The blood pressure measurement apparatus according to claim 1, further comprising:
a notification section that issues a notification of the teaching information.
